(19) United States
(12) Patent Application Publication
(10) Pub. No.: US 2013/0251814 A1
(43) Pub. Date: Sep. 26, 2013

(54) MAGNETIC NANODRUG FOR TREATING THROMBOSIS

(71) Applicants: Mu-Yi Hua, Tao-Yuan (TW); Hung-Wei Yang, Tao-Yuan (TW); Tony Wu, Taoyuan County (TW); Rung-Ywan Tsai, Kaohsiung City (TW); Yunn-Hwa Ma, Tao-Yuan (TW); Jyh-Ping Chen, Tao-Yuan (TW)

(72) Inventors: Mu-Yi Hua, Tao-Yuan (TW); Hung-Wei Yang, Tao-Yuan (TW); Tony Wu, Taoyuan County (TW); Rung-Ywan Tsai, Kaohsiung City (TW); Yunn-Hwa Ma, Tao-Yuan (TW); Jyh-Ping Chen, Tao-Yuan (TW)

(21) Appl. No.: 13/620,340

(22) Filed: Sep. 14, 2012

(30) Foreign Application Priority Data
Mar. 23, 2012 (TW) 101110199

(51) Int. Cl. A61K 9/51; A61K 31/4365; A61K 31/727; A61K 31/37; A61P 7/02; A61K 31/616; A61K 31/519; B82Y 5/00

(52) U.S. Cl. 424/497; 514/162; 514/301; 514/262.1; 514/56; 514/457; 977/906

(57) ABSTRACT

The present invention discloses a magnetic nanodrug for treating thrombosis, which comprises a core formed of magnetic nanoparticles, a shell enveloping the core and made of carboxyl-functionalized polyaniline, and a thrombosis-treatment drug covalently bonded to the shell. The magnetic nanodrug of the present invention is non-toxic to vascular endothelial cells, has superior stability, features superparamagnetism, and can be uniformly dissolved in water. Therefore, the magnetic nanodrug for treating thrombosis can be guided by an external magnetic field to concentrate on a specified region and increase the effect of thrombosis treatment.

### BACKGROUND OF THE INVENTION

1. Field of the Invention

The present invention relates to a magnetic nanodrug for treating thrombosis, particularly to a magnetic nanodrug able to fast concentrate on thrombus and treat thrombosis.

2. Description of the Related Art

Because of overnutrition and physical inactivity, modern people are likely to suffer from vascular narrowing or vascular occlusion. Vascular occlusion may cause cardiovascular diseases, regional apoplexy or even cerebral apoplexy. How to eliminate thrombus in the vessel is very important for the concerned patients.

At present, thrombosis is often treated with rt-PA (recombinant-tissue type plasminogen activator). Half life of rt-PA is only 20-30 minutes. Therefore, rt-PA is hard to achieve sufficient concentration and working time in vivo and less likely to dissolve thrombus effectively and promptly. Thus, the patients are usually treated with high density rt-PA. However, high density rt-PA risks massive bleeding. Accordingly, some carriers are used to transport the like drugs to prolong the in-vivo half life, such as liposome (Thromb Haemost, vol. 90, p. 64-70 (2003)) and polymeric nanoparticles (Biomaterials, vol. 29, p. 228-237 (2008)). However, the abovementioned technologies are unable to guide the drug to fast concentrate on thrombus.

Recently, magnetic nanoparticles have been widely used as the carriers of the related drugs. An external magnetic field is applied to guide the drug to concentrate on thrombus and increase the regional drug concentration, whereby is enhanced the effect of the drug. (Refer to Journal of Magnetism and Magnetic Materials, vol. 311, p. 376-378 (2007); Biomaterials, vol. 30, 5125-5130 (2009); Biomaterials, vol. 30, p. 3343-3351 (2009); and Thrombosis Research, vol. 121, p. 799-811 (2008).) Although the abovementioned technologies can guide drugs to thrombus, the applications thereof are limited by the efficiency and speed of drug releasing.

### SUMMARY OF THE INVENTION

In order to improve the current thrombosis treatment methods and overcome the problems of drug releasing in the abovementioned drug carrier technologies, the Inventors have been devoted to research and finally develop a magnetic nanodrug for treating thrombosis.

The present invention proposes a magnetic nanodrug for treating thrombosis, which exempts patients from surgery, and which is free of toxic chemicals, such as surfactants, dispersants and crosslinkers, wherein a thrombus-dissolving drug adheres to a magnetic nanocomposite. The nanodrug of the present invention features superparamagnetism. The nanocomposite has very large surface area. Thus, more drug molecules can adhere to each unit of nanocomposite. Thereby, an external magnetic field can promptly guide the magnetic nanodrug of the present invention to the thrombosis region to increase the regional drug concentration and enhance the therapy effect.

The magnetic nanodrug for treating thrombosis comprises a core formed of magnetic particles, a shell enveloping the core and made of carboxyl-functionalized polyaniline, and a thrombosis-treatment drug covalently bonded to the shell, wherein the thrombosis-treatment drug is bonded to the shell via EDC (1-ethyl-3-(3-dimethylaminepropyl) carbodiimidehydrochloride) and sulfo-NHS (N-hydroxysulfosuccinimide sodium salt). In one embodiment, the thrombosis-treatment drug is selected from a group consisting of rt-PA (recombinant-tissue type plasminogen activator), t-PA (tissue-type plasminogen activator), aspirin, Clopidogrel, Dipyridamole, Fraxiparine, Warfarin, and heparin.

The magnetic nanodrug of the present invention is thermostable and can be uniformly dissolved in water. Further, the magnetic nanodrug has superparamagnetism and can be guided to concentrate on a specified region by an external magnetic field. After stored at a temperature of 25° C. for 35 days, the magnetic nanodrug of the present invention still preserve 73% of the original activity. In vitro toxicity tests show that the nanocomposite SPAnH/MNPs, which is used to carry the thrombosis-treatment drug in the present invention, is non-toxic to vascular endothelial cells. In vitro thrombolysis tests for human blood clots show that the magnetic nanodrug guided by a magnetic field can faster dissolve thrombus than the conventional thrombolysis drugs. The present invention is an easy-to-fabricate, non-toxic, and magnetically-operable anti-thrombosis nanodrug having enhanced effect and high commercial potential.

### BRIEF DESCRIPTION OF THE DRAWINGS

FIG. 1 schematically shows the structure of a magnetic nanodrug for treating thrombosis according to one embodiment of the present invention;

FIG. 2 shows infrared spectra of MNPs and of SPAnH/MNPs according to one embodiment of the present invention;

FIG. 3 shows magnetic hysteresis curves of MNPs and SPAnH/MNPs according to one embodiment of the present invention;

FIG. 4 shows the SPAnH/MNPs bonding efficiency of rt-PA and the relative activity of rt-PA bonded to SPAnH/MNPs according to one embodiment of the present invention;

FIG. 5 shows the relative activities of rt-PA and the magnetic nanodrug stored at different temperatures according to one embodiment of the present invention;

FIG. 6 schematically shows an in-intro simulation apparatus according to one embodiment of the present invention;

FIG. 7 shows the results of in-vitro thrombolysis tests for the crude rt-PA, SPAnH/MNPs, and rt-PA/SPAnH/MNPs according to one embodiment of the present invention; and

FIG. 8 shows photographs of static in-vitro thrombolysis tests according to one embodiment of the present invention.

### DETAILED DESCRIPTION OF THE INVENTION

Below, embodiments are described in detail to make easily understood the present invention.

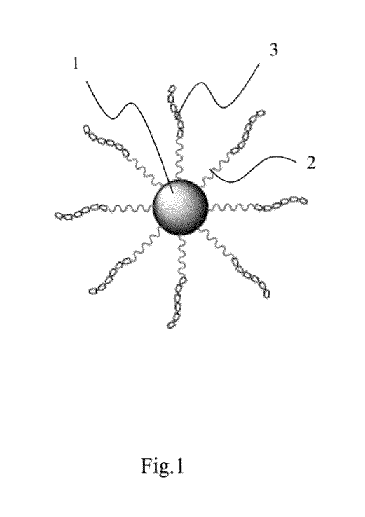

Refer to FIG. 1. The magnetic nanodrug for treating thrombosis comprises a core 1, a shell 2 and a drug layer 3. The core 1 is formed of magnetic particles having a diameter smaller than 10 nm. The shell 2 envelopes the core 1 and is made of carboxyl-functionalized polyaniline. The drug layer 3 contains a thrombosis-treatment drug covalently-bonded to the shell 2.

### Embodiment I

### A Method for Fabricating Neutral MNPs (Magnetic Nanoparticles) and a Method for Fabricating a Nanocomposite SPAnH (Superparamagnetic PAn (Polyaniline)-RCOOH)/MNPs

### Fabrication of MNPs:

A co-deposition method is used to fabricate the magnetic nanoparticles (MNPs) of ferrous ferric oxide (Fe₃O₄). Firstly, 0.7 g (4.32×10⁻³ mole) FeCl₃, 1.07 g (6.48×10⁻³ mole) FeCl₂.4H₂O and 400 ml of double distilled water are added into a three-necked bottle and agitated in a nitrogen-filled environment with a magnet for 5 minutes at an ambient temperature to completely dissolve FeCl₃ and FeCl₂.4H₂O. Next, 20 mL of 0.864N NaOH aqueous solution is added into the three-necked bottle, and the temperature of the solution is raised to a temperature of 80° C., whereby MNPs are formed.

### Separation of MNPs:

The reaction products are cooled down and vibrated with an ultrasonic vibrator to uniformly disperse MNPs in the aqueous solution. Next, the mixture solution is poured into a separating funnel, and a strong magnet is placed outside the funnel to attract the magnetic nanoparticles with the solution flowing away from the bottom of the funnel, whereby the solution and the magnetic nanoparticles are separated. Next, double distilled water is used to flush the magnetic nanoparticles repeatedly until the solution is neutralized and colorless. The obtained magnetic nanoparticles has a diameter of about 5-10 nm.

### Fabrication of SPAnH/MNPs:

Firstly, 10 ml of MNPs aqueous solution having a concentration of 6.4 mg/mL and 4 ml of an SPAnNa (poly[aniline-co-sodium N-(1-one-butyric acid) aniline]) aqueous solution having a concentration of 4.9 mg/mL are mixed uniformly. The mixed solution is vibrated with an ultrasonic vibrator, and a 0.5M HCl solution is dropped into the mixed solution. In an acidic environment, SPAnNa aggregates and envelops MNPs to form the nanocomposite SPAnH/MNPs with the core being MNPs and the shell being SPAnH. Next, the magnetic nanocomposite SPAnH/MNPs is separated from the solution and then dispersed in double distilled water to form an aqueous solution of SPAnH/MNPs for the later use. The magnetic nanocomposite SPAnH/MNPs has a diameter of about 15 nm.

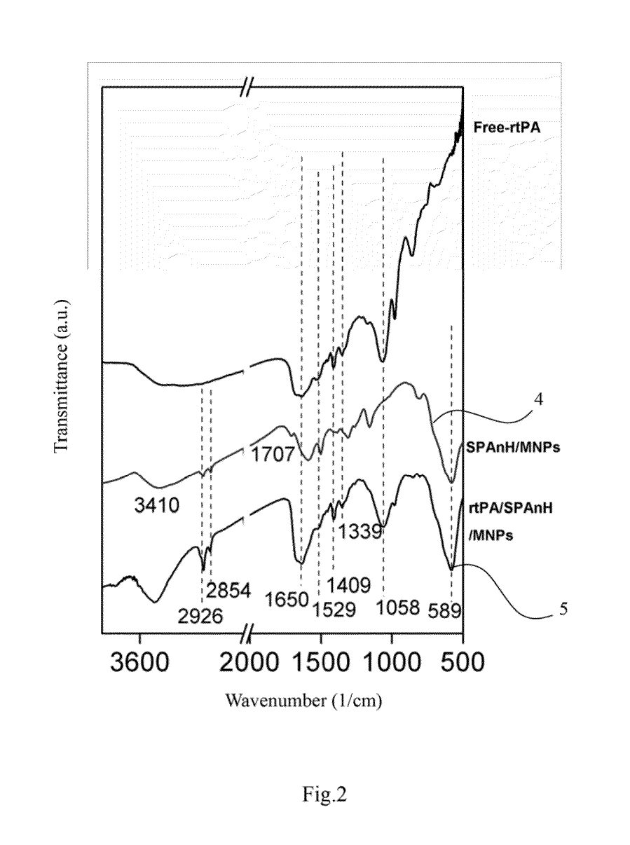

Refer to FIG. 2. Curve 4 and Curve 5 in FIG. 2 are respectively an infrared spectrum of SPAnH/MNPs and an infrared spectrum of rt-PA/SPAnH/MNPs. In the infrared spectrum of SPAnH/MNPs, there is a Fe—O stretching vibration (v_{Fe—O}) appearing at 586 cm⁻¹. In the infrared spectrum of the rt-PA/SPAnH/MNPs, there is also a Fe—O stretching vibration (v_{Fe—O}) appearing at 589 cm⁻¹; there are a saturation-symmetric stretching vibration and a saturation-asymmetric stretching vibration of C—H (v_{C—H}) respectively appearing at 2854 cm⁻¹ and 2926 cm⁻¹; and there are a C═O stretching vibration (v_{C═O}) and an O—H stretching vibration (v_{O—H}) of the functional group —COOH respectively appearing at 1707 cm⁻¹ and 3410 cm⁻¹. The abovementioned stretching vibrations indicate that MNPs is indeed enveloped by a layer of SPAnH. The shell of the magnetic nanodrug of the present invention has both carboxyl groups (—COOH) and amino groups (—NH), which favor attachment of drug molecules.

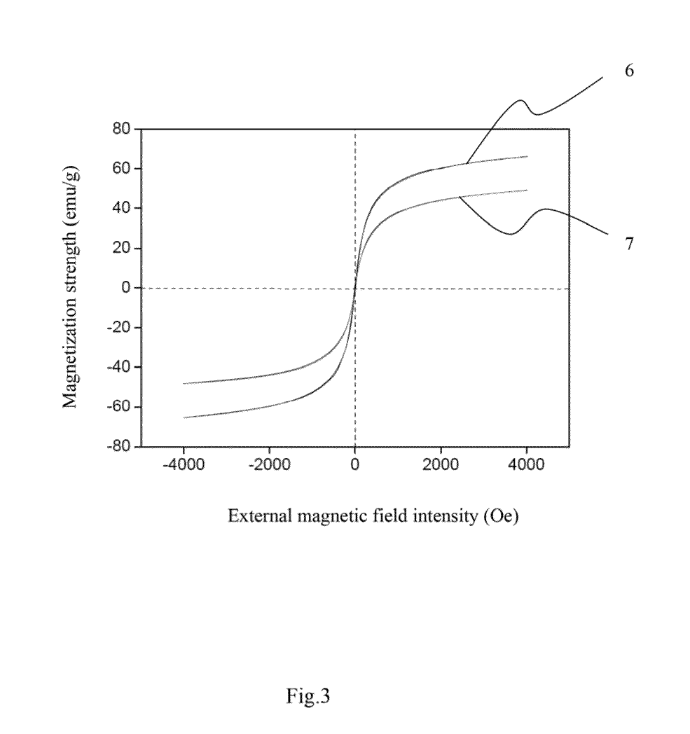

Refer to FIG. 3, wherein Curve 6 and Curve 7 are respectively magnetic hysteresis curves of MNPs and SPAnH/MNPs, which are obtained by SQUID (Superconducting Quantum Interference Device). The saturation magnetization of MNPs is found to be 66.2 emu/g. The saturation magnetization of MNPs normally increases with the magnetic field, and the magnetization curve passes through the origin, wherefore the magnetic nanoparticles MNPs of the present invention have the feature of superparamagnetism. It is found that the magnetic nanocomposite SPAnH/MNPs has a saturation magnetization of 37.6 emu/g and also features superparamagnetism.

### Embodiment II

### The Influence of SPAnH/MNPs Biotoxicity on Cellular Growth

### Preparation of Cell Lines:

Place HUVEC (Human Umbilical Vein Endothelial Cell) in a thermostatic incubator at a temperature of 37° C. and with 5% CO₂, and subculture HUVEC with a nutrient fluid M199 containing 2.2 mg/mL sodium hydrogen carbonate, 10% FBS (fetal bovine serum), 50 μg/mL gentamycin, 50 μg/mL penicillin, 50 μg/mL streptomycin, 25 U/mL heparin, and 30 μg/mL ECGS (Endothelial Cell Growth Supplement). Before subculture, a solution containing 1% gelatin is applied to the surfaces of the culture dishes. 2 mL of a solution containing 0.2 mg/mL Trypsin and 0.08 mg/mL EDTA is added to each culture dish. The culture dishes are placed still for 2 minutes to let the cells detach from the walls of the culture dishes. Next, place the nutrient fluid in centrifugal tubes, and process the centrifugal tubes at a speed of 1500 rpm and a temperature of 8° C. Then, remove the upper level liquid and uniformly disperse HUVEC in the nutrient fluid M199.

### Biotoxicity Test:

(1) Place 150 μL of a mixture liquid containing 10000 cells into the wells coated with 1% gelatin solution of a 96-well culture plate. Place the culture plate in a humidified incubator at a temperature of 37° C. and with 5% CO₂ to enable adherent growth of the cells. The next day, add to every well 50 μL of an SPAnH/MNPs solution where the magnetic nanocomposite SPAnH/MNPs are dispersed in the M199 nutrient fluid, and place the culture plate in a humidified incubator at a temperature of 37° C. and with 5% CO₂. One day after SPAnH/MNPs addition, observe the growth of the cells. Before cell counting, remove the nutrient fluid M199 from the wells, and then add 120 μL of XTT reaction fluid to every well. The culture plate is placed in an incubator for 3 hours to let reaction occur. Next, take 100 μL of post-reaction XTT reaction fluid from each well, and add them to a 96-well culture plate. Then, use an ELISA reader (BIO-TEK, model EL 808) to measure the OD value at a wavelength of 490 nm to estimate the biotoxicity of SPAnH/MNPs.

(2) Place 2 mL of a mixture liquid containing 20000 cells in culture dishes having a diameter of 3.5 cm and coated with 1% gelatin solution. Place the culture dishes in a humidified incubator at a temperature of 37° C. and with 5% CO₂ to enable adherent growth of the cells. The next day, add to every culture dish 100 μL at of an SPAnH/MNPs solution where the magnetic nanocomposite SPAnH/MNPs are dispersed in the M199 nutrient fluid. One day after SPAnH/MNPs addition, flush the contents of the culture dishes with 1 mL of HBSS (Hanks' Balanced Salt Solution). Next, add 1 mL of Live/Dead pigment to dye the cells for 30 minutes. Next, flush the contents with HBSS, and use a confocal microscope (Leica TCS SP2) to observe cell growth.

The relationship between the mitochondrial activity and the cultivation time in the case that HUVEC is cultivated in an environment containing 25-200 SPAnH/MNPs for 1-7 days. HUVEC cultivated in an environment free of SPAnH/MNPs is used as the control group.

After one day's cultivation, HUVEC cultivated in the environment containing SPAnH/MNPs is less than that of the control group. The higher the concentration of SPAnH/MNPs, the smaller the quantity of HUVEC. It may be because SPAnH/MNPs varies the property of the nutrient fluid and unfits HUVEC for the cultivation environment. In such a case, HUVEC gradually separates from the medium where it adheres and thus decreases in quantity.

HUVEC cultivated in an environment containing 25-150 μg/mL SPAnH/MNPs has a cell death rate of about 15% in the first day. However, the residual cells have adapted to the environment in the second day and start the adhering growth thereof. From the second day to the fifth day, the cells increase rapidly in a logistic growth mode. After the fifth day, the nutrient of the culture medium can no more afford the fast growth of cells. Thus, the cells enter a slow-growth stage until the seventh day. In the logistic-growth stage (from the second day to the fifth day), the growth rate of the cells of the experimental group is the same as that of the control group. Therefore, the nanocomposite SPAnH/MNPs has no obvious biotoxicity to HUVEC within the abovementioned range of concentration. In the case that the concentration of SPAnH/MNPs is increased to 200 μg/mL, the cells has a death rate of as high as 30% in the first day and increase slowly from the second day to the fifth day. This may be because the cells in the wells can no more tolerate and metabolize so high a concentration of SPAnH/MNPs.

The states of survival and growth of the cells are observed with an inverted confocal microscope. After one day's cultivation, the number of the cells in the SPAnH/MNPs-containing environment is less than that of the control group. However, the cells in the SPAnH/MNPs-containing environment increase with the accumulation of the cultivation days and grow in the same mode as the control group. The result matches the cellular growth curve and proves that the magnetic nanocomposite SPAnH/MNPs has no biotoxicity.

### Embodiment III

### A Method for Fabricating a Magnetic Nanodrug for Treating Thrombosis

Add 12 mg EDC (1-ethyl-3-(3-dimethylaminepropyl) carbodiimidehydrochloride) and 24 mg sulfo-NHS (N-hydroxysulfosuccinimide sodium salt) to a 0.5 M MES buffer solution having a pH value of 6.3 to form a reaction solution. Next, mix 0.2 mL of the reaction solution with 0.2 mL of an SPAnH/MNPs solution to form a mixture solution and vibrate the mixture solution at a temperature of 25° C. for one hour to obtain a resultant. Next, use a ferromagnetic magnet to separate the resultant from the solution, and use an MES buffer solution to flush it. Next, add a drug rt-PA (recombinant-tissue type plasminogen activator) to the resultant, and let them react at a temperature of 25° C. for 2 hours to make the molecules of the drug adhere to the resultant, whereby is obtained a reaction product. Next, use a ferromagnetic magnet to separate the reaction product and obtain a clarified liquid. Next, use a PBS buffer solution to flush the clarified liquid and perform a separation process to obtain the magnetic nanodrug for treating thrombosis (abbreviated as rt-PA/SPAnH/MNPs hereinafter) of the present invention.

### Embodiment IV

### Evaluating the Bonding Effects of Different Amounts of rt-PA to SPAnH/MNPs and the Relative Activities of rt-PA Bonded to SPAnH/MNPs

Use the method described in Embodiment III to respectively react 0.15 mg. 0.30 mg, 0.40 mg, 0.50 mg, 0.70 mg of rt-PA with the nanocomposite SPAnH/MNPs to test the bonding effects of different amounts of rt-PA and the activities of the magnetic nanodrugs formed thereby.

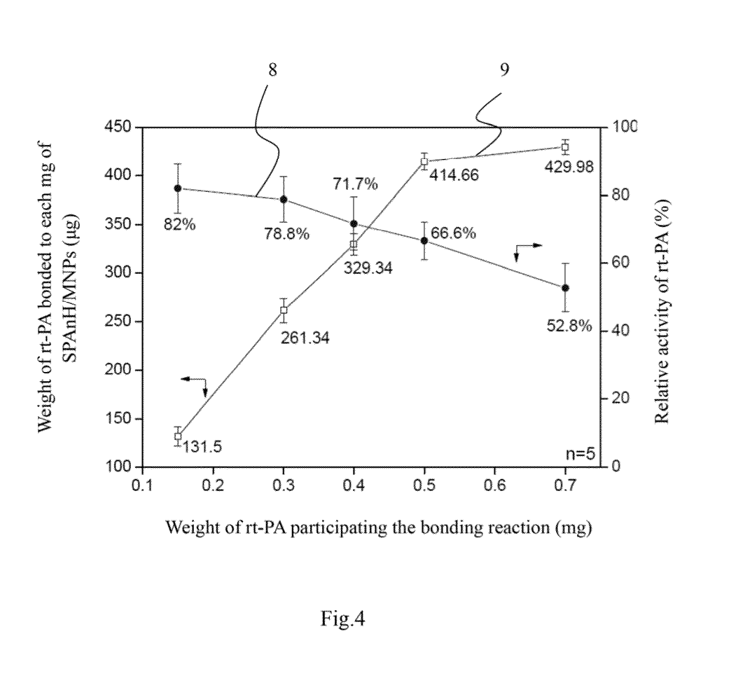

In the tests, the clarified liquids of the magnetic nanodrugs respectively formed with different amounts of rt-PA are uniformly mixed with a protein analysis pigment, and a spectrophotometer is used to detect the absorption value of a light beam having a wavelength of 595 nm so as to determine the amounts of free rt-PA. Next, use the amounts of free rt-PA to work out the amounts of rt-PA bonded to SPAnH/MNPs. The test results are shown in Table.1 and FIG. 4. When the added rt-PA is increased to 0.70 mg, the amount of rt-PA bonded to SPAnH/MNPs no more increases proportionally but gradually becomes stable. It means that rt-PA has been bonded to all the carboxyl groups of SPAnH/MNPs. In fact, too much rt-PA bonded to SPAnH/MNPs would cause a steric hindrance effect, which will hinder a portion of rt-PA from participating in reaction. Therefore, it is preferred that 1 mg of SPAnH/MNPs has 276.2 μg of rt-PA bonded thereto.

TABLE 1
shows the bonding effects of different amounts of rt-PA to
SPAnH/MNPs and the activities of rt-PA bonded to SPAnH/MNPs

| Weight | Weight of rt-PA | Relative activity of rt-PA |
|---|---|---|
| of rt-PA | bonded to | bonded to |
| (mg) | SPAnH/MNPs (μg) | SPAnH/MNPs (%) |
| 0.15 | 131.50 | 82.0 |
| 0.30 | 261.34 | 78.8 |
| 0.40 | 329.34 | 71.7 |
| 0.50 | 414.66 | 66.6 |
| 0.70 | 429.98 | 52.8 |

### Embodiment V

### Stability Tests of the Magnetic Nanodrug at Different Temperatures

Respectively store the magnetic nanodrugs of the present invention at temperatures of 4° C. and 25° C. for 1-35 days, and then use a protein analysis method to detect the residual activities of the magnetic nanodrugs to evaluate the stability of the magnetic nanodrugs.

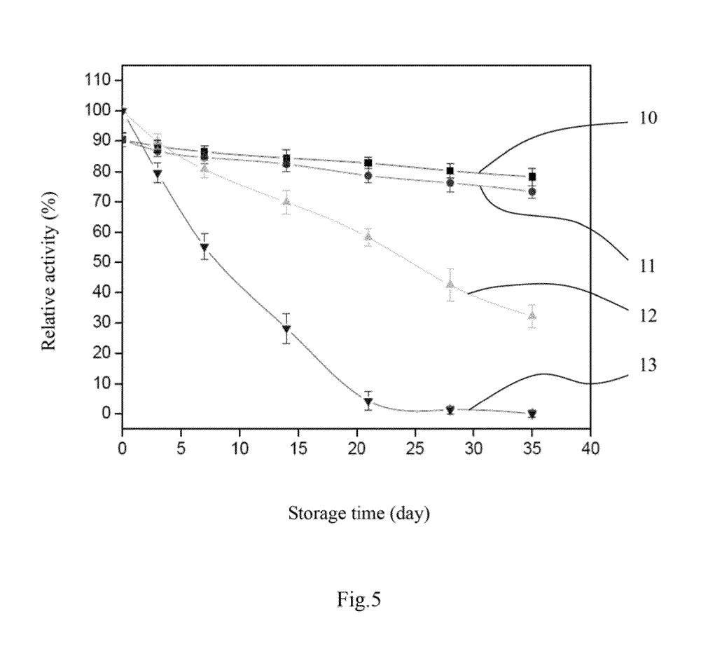

Refer to FIG. 5. The crude rt-PA stored at a temperature of 25° C. has lost all the activity on the 21^{st} day. The crude rt-PA stored at a temperature of 4° C. can keep about 32% of the activity on the 35^{th} day. The magnetic nanodrug stored at a temperature of 25° C. still keeps about 73% of the activity on the 35^{th} day. The magnetic nanodrug stored at a temperature of 4° C. keeps about 78% of the activity on the 35^{th} day. Bonding rt-PA to the magnetic nanocomposite may decrease the activity, but it can prevent rt-PA from ionization and make the molecular morphology of rt-PA less likely to change. Therefore, bonding rt-PA to SPAnH/MNPs can increase the stability of rt-PA and extend the expiry time thereof.

### Embodiment VI

### Static In-Vitro Thrombolysis Tests of the Magnetic Nanodrug for Treating Thrombosis

### Preparation of Blood Clots:

Seal one end of a PE tube having a diameter of 5 mm and a length of 35 mm, and inject 0.5 mL of human blood into the PE tube and seal the other end immediately. Place the blood-containing PE tube in an environment at a temperature of 37° C. to undertake a coagulation reaction for 24 hours. Then, take out the coagulated blood strip, and cut the blood strip into 1 mm×2 mm blood clots.

### Preparation of Plasma:

Place 4.5 mL of human blood and 0.5 mL of sodium citrate in a centrifugal tube, and centrifugally process the mixture at a speed of 6000 rpm for 20 minutes. The supernatant in the tube is exactly the platelet poor plasma (abbreviated as PPP hereinafter).

### Preparation of a Standard Curve:

Add to each of 5 mL sample bottles a blood clot, 800 μL of physiological saline and 100 μL of PPP. Respectively add 100 μL of 0.2, 0.4, 0.6, 0.8 and 1.0 mg/mL rt-PA to the sample bottles. Vibrate the sample bottles at a speed of 150 rpm and a temperature of 37° C. for 30 minutes to undertake reaction. Take out the supernatants in the sample bottles, and use a spectrophotometer to detect the absorption values of a light by hematin to establish a standard curve, wherein the light has a wavelength of 405 nm.

### Thrombolysis Tests:

Add to each of 5 mL sample bottles a blood clot, 800 μL of physiological saline and 100 μL of PPP. Respectively add 30 μg of rt-PA and 61.9 μg of the magnetic nanodrug of the present invention to the sample bottles. Vibrate the sample bottles at a speed of 150 rpm and a temperature of 37° C. for 30 minutes under an external magnetic field or without any external magnetic field. Take out the supernatants in the sample bottles, and use a spectrophotometer to detect the absorption values that a light having a wavelength of 405 nm is absorbed by hematin, and compare the results with the standard curve to determine the thrombolysis effects.

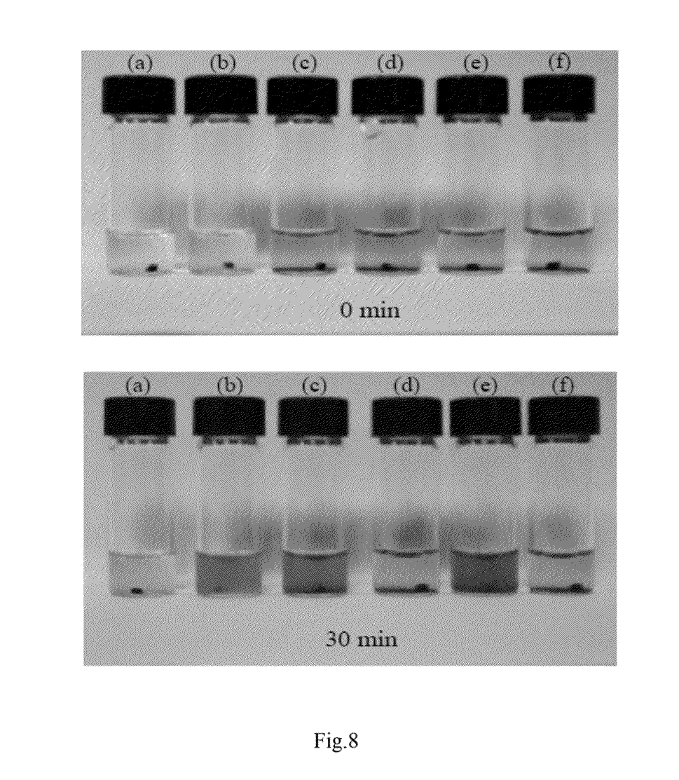

Refer to FIG. 8, wherein the upper photograph shows the pre-reaction states, and the lower photograph shows the post-reaction states. In FIG. 8, (a) shows the control group where no drug is added; (b) shows the thrombolysis state achieved by rt-PA; (c) shows the thrombolysis state achieved by the magnetic nanodrug of the present invention without any external magnetic field; (d) shows the thrombolysis state achieved by the magnetic nanocomposite of the present invention without any external magnetic field; (e) shows the thrombolysis state achieved by the magnetic nanodrug of the present invention under an external magnetic field; (f) shows the thrombolysis state achieved by the magnetic nanocomposite of the present invention under an external magnetic field.

Without any external magnetic field, the absorption value for the magnetic nanodrug of the present invention is 0.558. According to the standard curve, the absorption value of 0.558 is converted into a reacted drug amount of 41.03 μg and a drug reaction ratio of 66.28%. From the photographs, it is observed that the color of hematin in Case (c) wherein the magnetic nanodrug of the present invention is used without any external magnetic field is darker than that of Case (b) wherein the crude rt-PA is used. Therefore, the magnetic nanodrug of the present invention participates in reaction in a higher proportion.

When an external magnetic field is applied to the bottoms of the bottles, the magnetic nanodrug of the present invention is attracted to the bottom and surrounds the blood clot. When the blood clot is vibrated to swing and roll, the blood clot contacts further more of the magnetic nanodrug of the present invention. Thus, the plasminogen on the surface of the blood clot is converted into plasmin faster. Therefore, in Case (e), the drug reaction ratio increases to as high as 86.12%, which is corresponding to an absorption value of 0.725 and a reacted drug amount of 53.31 μg. The color of hematin generated by the thrombolysis reaction in Case (e) is darker than the colors of Case (b) and Case (c). Therefore, the magnetic nanodrug of the present invention has further higher thrombolysis performance under an external magnetic field.

No matter whether there is an external magnetic field, the magnetic nanocomposite SPAnH/MNPs cannot dissolve the blood clot.

### Embodiment VII

### Dynamic In-Vitro Thrombolysis Tests of the Magnetic Nanodrug for Treating Thrombosis

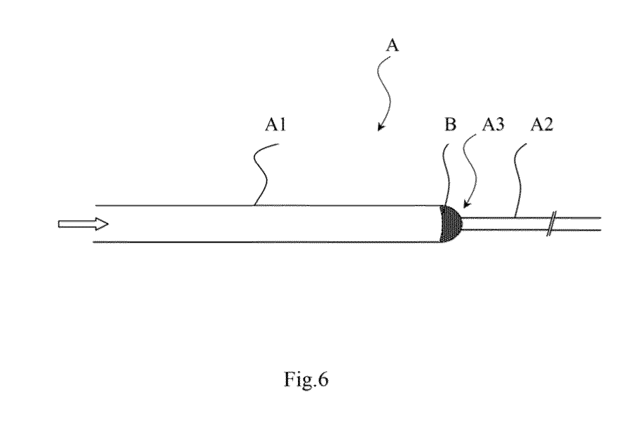

Refer to FIG. 6. In this embodiment, the methods for preparing blood clots and plasma are the same as those of Embodiment VI. A wriggle-type pump (not shown in the drawing) is pivotally coupled to a glass dropper A to simulate the flowing and thrombolysis of the magnetic nanodrug in vitro and evaluate the performance thereof. In this embodiment, the flow rate of the wriggle-type is set to be 0.18 mL/min. A PPP/physiological saline solution (the volume ratio thereof=1/4) is transported to the cylindrical portion A1 of the glass dropper A. The left end of the glass dropper A is a pressure-relief opening, and the right end simulates the embolism region of a blood vessel. A 2 mm×5 mm blood clots used to block the taper portion A3 of the glass dropper A. After the flow has reached a steady state, 100 mL rt-PA (250 μg), 100 mL magnetic nanodrug of the present invention (250 μg) and 100 mL SPAnH/MNPs are respectively slowly injected into different glass droppers A from the position indicated by an arrow (10 cm behind the blood clot). A powerful magnet is used to guide the magnetic nanodrug and the magnetic nanocomposite to the surroundings of the blood clots. Then, count the time interval for dissolving the blood clots to such an extent that the blood clots can completely flow out of the tip A2 at the right.

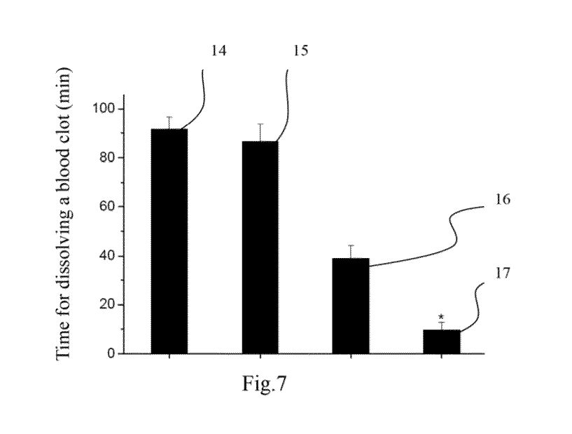

Refer to FIG. 7, wherein Bars 14-17 respectively represent the results of the thrombolysis tests of the control group, SPAnH/MNPs of the present invention, the crude rt-PA, and the magnetic nanodrug of the present invention. As the crude rt-PA cannot flow to the blood clot rapidly and convert the plasminogen into plasmin in a short time, it takes 39 minutes to dissolve the blood clot to such an extent that the blood clot can flow out of the outlet of the tip A2. Guided by an external magnetic field, the magnetic nanodrug of the present invention can reach the blood clot faster. The magnetic nanodrug of the present invention takes only about 9 minutes to make the blood clot flow out of the tip A2. Therefore, the magnetic nanodrug of the present invention can greatly reduce the time for dissolving a blood clot.

In conclusion, the magnetic nanodrug for treating thrombosis of the present invention can be fabricated in a simple way; each mg of the magnetic nanoparticle allows 0-430 μg rt-PA to bond thereto; the rt-PA bonded to the magnetic nanoparticles keeps 52.8-95.6% of the activity; the activities of the magnetic nanodrug of the present invention respectively stored at temperatures of 4° C. and 25° C. are about 46.0% and 73.3% higher than the activities of the crude rt-PA stored under the same conditions; the thrombolysis effect is increased 19.8% by the magnetic nanodrug of the present invention under an external magnetic field in a static in-vitro thrombolysis test; the magnetic nanodrug of the present invention saves about 30 minutes of the thrombus-dissolving time under an external magnetic field in a dynamic in-vitro thrombolysis test; the magnetic nanodrug of the present invention is non-toxic to vascular endothelial cells, has superior stability, features superparamagnetism, and can be uniformly dissolved in water. Therefore, the magnetic nanodrug for treating thrombosis can be guided by an external magnetic field to concentrate on a specified region and increase the effect of thrombosis treatment.

What is claimed is:

1. A magnetic nanodrug for treating thrombosis, comprising
a core formed of magnetic nanoparticles having a diameter of less than 10 nm;
a shell enveloping said core and made of carboxyl-functionalized polyaniline; and
a thrombosis-treatment drug covalently bonded to said shell.

2. The magnetic nanodrug for treating thrombosis according to claim 1, wherein said magnetic nanoparticles are made of a material selected from a group consisting of ferric oxide, ferrous ferric oxide, and nickel.

3. The magnetic nanodrug for treating thrombosis according to claim 1, wherein said carboxyl-functionalized polyaniline is expressed by a molecular formula: 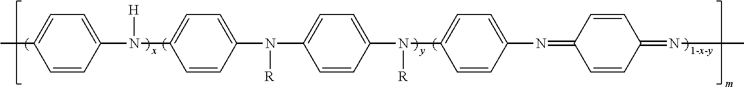
wherein —R is a functional group selected from a set consisting of —H, —COR₁COO⁻, —COR₁COOH, —COR₁COOLi, —COR₁COONa, —COR₁COOK, —COR₁COONH₄⁺, —COR₁CONH₂, —R₁COOH, —R₁COOLi, —R₁COONa, —R₁COOK, —R₁COONH₄⁺ and —R₁CONH₂; and
wherein R₁ is selected from a set consisting of alkyls containing 2-4 carbon atoms and alkenyls containing 2-4 carbon atoms.

4. The magnetic nanodrug for treating thrombosis according to claim 2, wherein said carboxyl-functionalized polyaniline is expressed by a molecular formula: 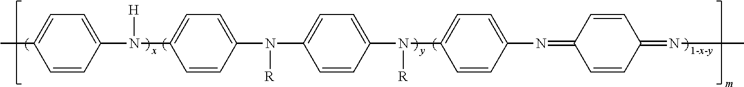
wherein —R is a functional group selected from a set consisting of —H, —COR₁COO⁻, —COR₁COOH, —COR₁COOLi, —COR₁COONa, —COR₁COOK, —COR₁COONH₄⁺, —COR₁CONH₂, —R₁COOH, —R₁COOLi, —R₁COONa, —R₁COOK, —R₁COONH₄⁺ and —R₁CONH₂; and
wherein R₁ is selected from a set consisting of alkyls containing 2-4 carbon atoms and alkenyls containing 2-4 carbon atoms.

5. The magnetic nanodrug for treating thrombosis according to claim 1 having a diameter of 25-50 nm.

6. The magnetic nanodrug for treating thrombosis according to claim 1, wherein said thrombosis-treatment drug is selected from a group consisting of rt-PA (recombinant tissue-type plasminogen activator), t-PA (tissue-type plasminogen activator), aspirin, Clopidogrel, Dipyridamole, Fraxiparine, Warfarin and heparin.

7. The magnetic nanodrug for treating thrombosis according to claim 5, wherein molecules of said thrombosis-treatment drug are bonded to said shell at a temperature of 20-25° C.
